(19) United States
(12) Patent Application Publication
(10) Pub. No.: US 2021/0225517 A1
(43) Pub. Date: Jul. 22, 2021

(54) PREDICTIVE MODEL FOR ADVERSE PATIENT OUTCOMES

(71) Applicant: THE CLEVELAND CLINIC FOUNDATION, Cleveland, OH (US)

(72) Inventors: Orkun Baloglu, Cleveland, OH (US); Kristopher Kormos, Cleveland, OH (US); Samir Latifi, Cleveland, OH (US)

(21) Appl. No.: 17/153,772

(22) Filed: Jan. 20, 2021

Related U.S. Application Data
(60) Provisional application No. 62/964,040, filed on Jan. 21, 2020.

(51) Int. Cl. G16H 50/30; G16H 10/60; G16H 20/40; G16H 50/70

(52) U.S. Cl. CPC G16H 50/30; G16H 50/70; G16H 20/40; G16H 10/60

(57) ABSTRACT

Systems and methods are provided for predicting an adverse patient outcome. A set of biometric parameters associated with a patient are monitored and at least one electronic health records (EHR) parameter is retrieved from an EHR database. A set of categorical parameters are generated from the set of biometric parameters and one or more EHR parameters according to a predefined rule set. A score, representing a risk that a patient will experience an adverse patient outcome, is generated from the set of categorical parameters.

### RELATED APPLICATIONS

This application claims priority from U.S. Provisional Application No. 62/964,040, filed 21 Jan. 2020, the subject matter of which is incorporated herein by reference in its entirety.

### TECHNICAL FIELD

This invention relates to medical diagnostic systems, and more particularly, to a predictive model for adverse patient outcomes.

### BACKGROUND

Severity of illness (SOI) of critically ill patients is prone to change rapidly and assessment of SOI requires frequent evaluation of clinical and laboratory data. Stratification of intensive care unit (ICU) patients by SOI may allow clinicians to triage their attention, anticipate further physiological deterioration, and mobilize ICU resources. Moreover, lack of recognition of clinical deterioration is considered among the important factors contributing to cardiac arrest (CA) events in pediatric ICUs.

### SUMMARY

In accordance with one aspect of the invention, a method is provided for predicting an adverse patient outcome. A set of biometric parameters associated with a patient are monitored and at least one electronic health records (EHR) parameter is retrieved from an EHR database. A set of categorical parameters are generated from the set of biometric parameters and one or more EHR parameters according to a predefined rule set. A score, representing a risk that a patient will experience an adverse patient outcome, is generated from the set of categorical parameters.

In accordance with another aspect of the invention, a system is provided. A biometric monitor interface receives data from one or more monitoring systems monitoring a set of biometric parameters associated with a patient. A network interface retrieves at least one electronic health records (EHR) parameter from an EHR database. A feature extractor generates a set of categorical parameters from the set of biometric parameters and the EHR parameter according to a predefined rule set. A predictive model generates a score, representing a risk that a patient will experience an adverse patient outcome, from at least the set of categorical parameters.

In accordance with a further aspect of the invention, a method is provided for predicting an adverse patient outcome. A set of biometric parameters associated with a patient are monitored and at least one electronic health records (EHR) parameter is retrieved from an EHR database. A set of categorical parameters are generated from the set of biometric parameters and one or more EHR parameters according to a predefined rule set. At periodic intervals, a score, representing a risk that a patient will experience an adverse patient outcome, is generated from the set of categorical parameters to provide a time series of scores. An extremum of the time series of scores over a predefined interval of time is selected, and a parameter representing the risk that the patient will experience the adverse patient outcome is generated from the selected extremum.

### BRIEF DESCRIPTION OF THE DRAWINGS

FIG. 1 illustrates a system implementing a model for predicting adverse patient outcomes in accordance with an aspect of the present invention;

FIG. 2 illustrates a software-implemented system, implemented on one or more computer readable media, for employing a predictive model for assessing the risk of an adverse patient outcome for a patient in accordance with an aspect of the present invention;

FIG. 3 is a method for predicting an adverse patient outcome for a patient;

FIG. 4 illustrates a method for determining a parameter representing a risk that a patient will experience an adverse patient outcome; and

FIG. 5 is a schematic block diagram illustrating an exemplary system of hardware components.

### DETAILED DESCRIPTION

An “adverse patient outcome” is an event for an intensive care unit (ICU) patient that results in initiation or withdrawal of therapeutic intervention outside of the planned scope of the patient's care or death. Examples of therapeutic intervention that can be necessitated by such an event include administration of vasoactive medications, non-invasive positive pressure ventilation, invasive positive pressure ventilation (i.e., endotracheal intubation), extracorporeal membrane oxygenation, surgical intervention, mechanical circulatory support (e.g., extracorporeal membrane oxygenation, ventricular assist device, intra-aortic balloon pump etc.), use of inhaled nitric oxide, renal replacement therapies, organ transplant, tracheostomy, decompressive craniectomy, antibiotic administration, blood product administration, intravascular thrombectomy or thrombolysis, implantation of a pacemaker, or an increase in the length of time a patient remains in the ICU. Examples of adverse patient outcomes include death, cardiac arrest, thrombosis, neurological disability, advent or worsening of respiratory distress, renal failure, and similar outcomes.

A “predictive model,” as used herein, is a mathematical or machine learning model that predicts a parameter associated with adverse patient outcomes.

Identifying a likely adverse patient outcome prior to its occurrence, even five minutes before, provides a time window for clinicians to mobilize appropriate resources and intervene to prevent or mitigate the adverse outcome. This would hopefully result in improved survival and decreased neurological morbidities in patients, particularly in intensive care unit (ICU) patients. Accordingly, the present invention provides a predictive model for patient risk assessment that is for adult and pediatric patients with scored components that are objectively measured and are being automatically collected by an electronic health records system. This allows for a continuous, real-time risk assessment.

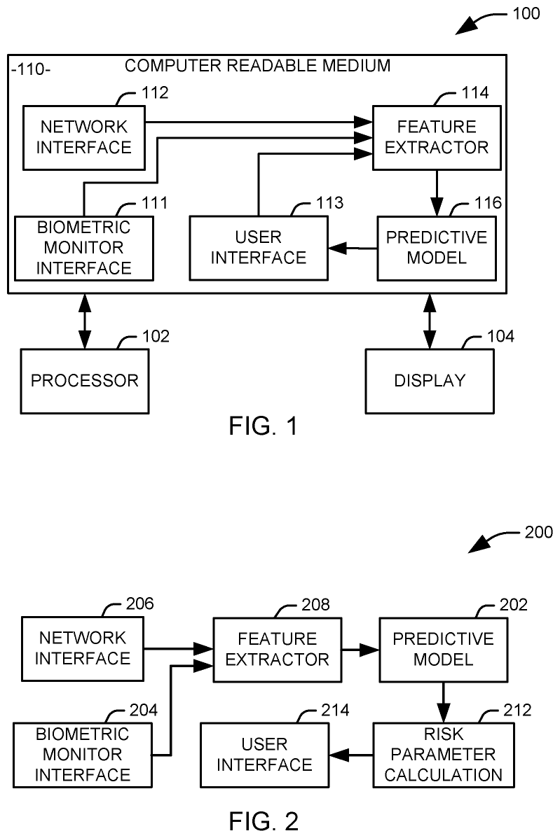

FIG. 1 illustrates a system 100 for employing a predictive model for assessing the risk of an adverse patient outcome for a patient in accordance with an aspect of the present invention. The system 100 includes a processor 102, a display 104, and a non-transitory computer readable medium 110 storing computer readable instructions, executed by the processor 102. The executable instructions stored on the non-transitory computer readable medium 110 include a biometric monitor interface 111 that receives data from one or more monitoring systems tracking biometric parameters for the patient. Monitored patent parameters can include heart rate, bispectral index, values extracted from electrocardiograms and electroencephalograms, arterial blood pressure, respiratory rate, intracranial pressure, central venous pressure, left and right atrial pressure, pulmonary artery and pulmonary artery wedge pressure, ejection fraction, shortening fraction, cardiac index, urine output, serum electrolytes, serum lactic acid, blood glucose, hemoglobin, platelet, and white blood cell counts, parameters derived from a coagulation profile, results from laboratory tests for renal and liver function, peripheral arterial oxyhemoglobin saturation, as measured by pulse oximetry, cerebral and microcirculatory blood flow and oxygen saturations derived from near infrared spectroscopy, venous oxyhemoglobin saturation, end-tidal carbon dioxide levels, temperature, settings for a mechanical ventilator, including positive end expiratory pressure (PEEP), fraction of inspired oxygen (FiO2), Peak and Plateau Inspiratory Pressure etc.) or a mechanical circulatory support device, such as an extracorporeal membrane oxygenation (ECMO) system, a ventricular assist device, or an intra-aortic balloon pump.

The executable instructions further include a network interface 112 via which the system 100 communicates with other systems (not shown) via a network connection, for example, an Internet connection and/or a connection to an internal network. In the illustrated example, the other systems can include an electronic health records (EHR) system that stores medical information for the patient, and the network interface 112 can include an application program interface (API) (not shown) for communicating with the EHR system. Data retrieved from the EHR can include, for example, demographics, such as age and gender, a primary diagnosis at the time of ICU admission, any occurrence of cardiac arrest during the ICU stay, the length of the ICU stay, blood gas analysis results, serum lactate values, the use of any vasoactive medications for the patient, and a type of any respiratory support provided to the patient. It will be appreciated that, in some implementations, the monitoring systems can communicate with the system 100 via a local or wide-area network connection, and that, in this instance, the network interface 112 and the biometric monitor interface 111 may share some or all of their components. Further, where patient data is not available from the EHR, relevant information for the patient can be entered via an appropriate user interface 113.

Information retrieved via the biometric monitor interface 111 and the network interface 112 is provided to a feature extractor 114 that extracts a plurality of features for use at a predictive model 116. In one implementation, the feature extractor 114 can determine descriptive statistics, such as measures of central tendency (e.g., median, mode, arithmetic mean, or geometric mean) and measures of deviation (e.g., range, interquartile range, variance, standard deviation, etc.) of time series of the biometric parameter. Additionally or alternatively, the biometric parameters and the data extracted from the EHR can be used to assign a plurality of categorical parameters to the patient according to various rule sets. For example, the patient can be assigned a categorical parameter representing the presence of hypothermia if the body temperature is below a threshold value. In one implementation, the categorical parameters can include the presence or absence of hypothermia, hyperthermia, elevated serum lactate, hypoxemia, respiratory acidosis, the use of vasopressors for the patient, the use of non-invasive positive pressure respiratory support for the patient, the use of an artificial airway for the patient, tachycardia, bradycardia, tachypnea, bradypnea, hypotension, and hypertension.

It will be appreciated that the rule sets used for the assignment of clinical parameter can be more complex, for example with thresholds or ranges for a given parameter that vary according to the values of other biometric parameters or EHR data or the comparison of multiple biometric parameters or EHR values to ranges, thresholds, and, for categorical data, individual values. For example, a tachycardia parameter can be assigned if the heart rate of the patient exceeds a threshold value that depends on an age of the patient. Accordingly, the features extracted by the feature extractor 114 can include continuous or categorical values provided by the biometric monitor interface 111 and the network interface 112, descriptive statistics generated from time series of these values, or categorical parameters generated via the application of defined rule sets to these values.

The predictive model 116 can utilize one or more pattern recognition algorithms, each of which analyze the extracted features or a subset of the extracted features to classify the patients into one of the plurality of classes and provide this information to the display 104. Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. For rule-based models, such as decision trees, domain knowledge, for example, as provided by one or more human experts, can be used in place of or to supplement training data in selecting rules for classifying a patient using the extracted features. Any of a variety of techniques can be utilized for the classification algorithm, including support vector machines (SVMs), regression models, self-organized maps, fuzzy logic systems, data fusion processes, boosting and bagging methods, rule-based systems, or artificial neural networks.

For example, a support vector machine (SVM) classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used. In the illustrated implementation, the classifier includes one or both of a support vector machine and a random forest classifier. While the illustrated implementation utilizes one or more classifiers to categorize the patient, it will be appreciated that a regression model or similar approach can be employed to give a continuous, as opposed to a categorical output.

In the illustrated implementation, the predictive model 116 uses a rule-based classifier to assign a score to a patient representing a risk that the patient will experience an adverse patient outcome given the extracted features. In one example, each of fourteen categorical parameters provided from the feature extractor 114 can be assigned a categorical value, and a set of scoring rules can be applied using these values to generate a score for the patient. It will be appreciated that the scoring rules can include products and sums of weighted values representing the parameters as well as exponential powers of the products and sums of weighted values representing the parameters, such that the score is a non-linear function of the values. In one example, the risk represented by the score can vary with the length of the patient's stay in the ICU, such that a unit increase in the score represents a different increase in risk to the patient depending on the duration of the ICU stay. The generated score can be provided to the user at the display 104 via the user interface 113.

FIG. 2 illustrates a software-implemented system 200, implemented on one or more computer readable media (not shown), for employing a predictive model 202 for assessing the risk of an adverse patient outcome for a patient in accordance with an aspect of the present invention. In the specific implementation described herein, the adverse patient outcome is cardiac arrest. The system 200 includes a biometric monitor interface 204 that receives data from one or more monitoring systems tracking biometric parameters for the patient. In the illustrated implementation, monitored patent parameters can include, for example, heart rate, arterial blood pressure, respiratory rate, serum electrolytes, peripheral arterial oxyhemoglobin saturation, as measured by pulse oximetry, cerebral and microcirculatory blood flow and oxygen saturations derived from near infrared spectroscopy, end-tidal carbon dioxide levels, body temperature, settings for a mechanical ventilator, including positive end expiratory pressure (PEEP), fraction of inspired oxygen (FiO2), Peak and Plateau Inspiratory Pressure, etc.).

The executable instructions further include a network interface 206 via which the system 200 interfaces with appropriate hardware to communicate with an electronic health records (EHR) system that stores medical information for the patient. Data retrieved from the EHR can include, for example, demographics, such as age and gender, a primary diagnosis at the time of ICU admission, any occurrence of cardiac arrest during the ICU stay, the length of the ICU stay, blood gas analysis results, serum lactate values, the use of any vasoactive medications for the patient, and a type of any respiratory support provided to the patient.

Information retrieved via the biometric monitor interface 204 and the network interface 206 is provided to a feature extractor 208 that extracts a plurality of features for use at the predictive model 202. In the illustrated implementation, the feature extractor 208 can assign a plurality of categorical parameters to the patient according to various rule sets. For example, the patient can be assigned a categorical parameter representing the presence of hypothermia if the body temperature is below a threshold value. In another example, a tachycardia parameter can be assigned a first value when a heart rate of a patient exceeds a threshold value and a second value when the heart rate of the patient does not exceed the threshold value, and the threshold value is selected according to an age of the patient In the illustrated implementation, the categorical parameters can include parameters representing the presence or absence of hypothermia, hyperthermia, elevated serum lactate, hypoxemia, respiratory acidosis, the use of vasopressors for the patient, the use of non-invasive positive pressure respiratory support for the patient, the use of an artificial airway for the patient, tachycardia, bradycardia, tachypnea, bradypnea, hypotension, and hypertension.

The extracted features are then provided to the predictive model 202 to calculate a score representing a risk that a patient will experience cardiac arrest. In the illustrated, each categorical parameter can be represented as a numerical value based upon its assigned value, and a non-linear weighted combination of these values can be used to determine the score. The scores are calculated at periodic intervals and provided to a risk parameter calculation component 212 that determines a risk parameter representing a risk that the patient will experience cardiac arrest from the calculated scores. The determined risk parameter is displayed to a user via a user interface 214.

In one implementation, an extreme score (e.g., highest or lowest score) is selected from a predefined window of time and the risk parameter is determined from the extreme score. For example, a continuous risk parameter could be determined as a function of the extreme score, an ordinal ranking of patients could be achieved by comparing the risk across patients, or a categorical parameter could be determined by comparing the extreme score to one or more threshold values. In one example, a maximum score within four-hour time interval immediate preceding determination of the risk parameter was found to achieve promising performance in discriminating patients regarding occurrence of cardiac arrest and was significantly associated with higher odds of cardiac arrest in ICU patients. For one threshold value, a classification of patients into “normal” and “enhanced risk” groups resulted in 82.1% and 83.2% of sensitivity and specificity, respectively.

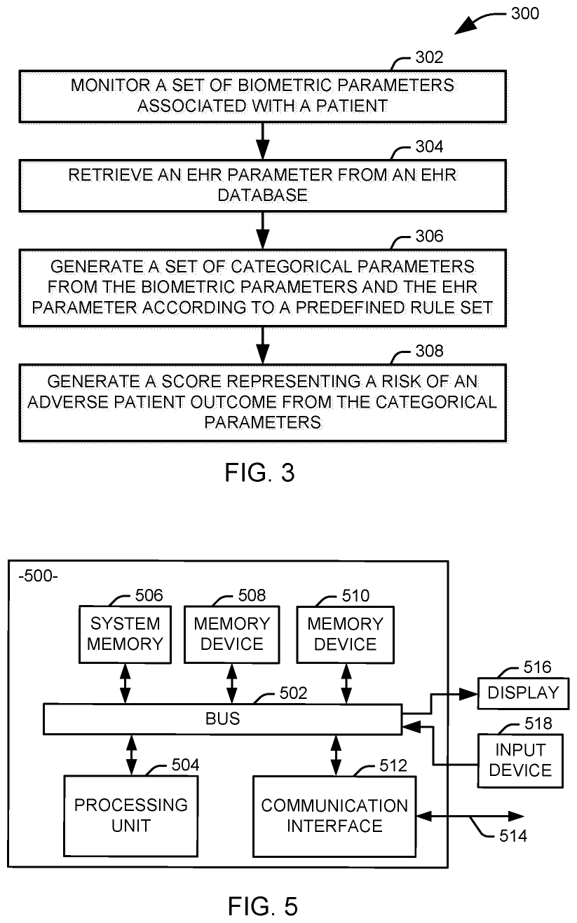

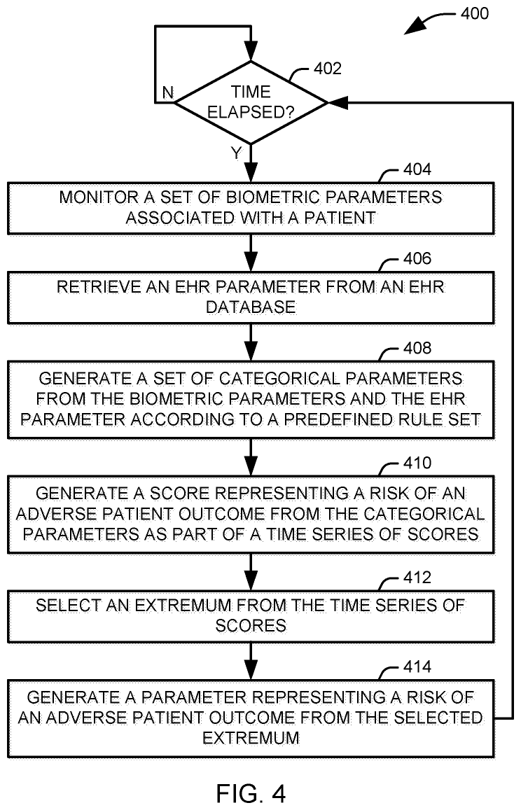

In view of the foregoing structural and functional features described above, methods in accordance with various aspects of the present invention will be better appreciated with reference to FIGS. 3 and 4. While, for purposes of simplicity of explanation, the methods of FIGS. 3 and 4 are shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a method in accordance with an aspect the present invention.

FIG. 3 is a method 300 for predicting an adverse patient outcome for a patient. At 302, a set of biometric parameters associated with a patient are monitored. In one example, the set of biometric parameters include at least two of a heart rate, arterial blood pressure, peripheral arterial oxyhemoglobin saturation, and body temperature. At 304, at least one electronic health records (EHR) parameter is retrieved from an EHR database. It will be appreciated that the EHR parameters can include any stored parameters associated with the condition, treatment, and medical history of the patient, and can include, for example, demographics, such as age and gender, a primary diagnosis at the time of ICU admission, any occurrence of cardiac arrest during the ICU stay, the length of the ICU stay, blood gas analysis results, serum lactate values, the use of any vasoactive medications for the patient, and a type of any respiratory support provided to the patient.

At 306, a set of categorical parameters are generated from the set of biometric parameters and the at least one EHR parameter according to a predefined rule set. In one implementation, some of the biometric parameters that are continuous or discrete with a large number of possible values can be compared to sets of threshold values to convert the biometric parameters into categorical parameters. In this implementation, the sets of threshold values can be variable based upon values of the EHR parameters. For example, one categorical parameter can represent the presence or absence of tachycardia, with a heart rate of the patient compared to a threshold value to assign a first value, representing the presence of tachycardia, or a second value, representing the absence of tachycardia. The threshold values can include several values based on age, for infants, additional values for young children, and a threshold value for teens and adults.

In another example, one categorical parameter can represent the presence or absence of hypothermia, with a body temperature of the patient compared to a threshold value to assign a first value, representing the presence of hypothermia, or a second value, representing the absence of hypothermia. In this instance, the threshold value, by default, is constant across all patients. Other parameters can represent, for example, the presence of respiratory acidosis or elevated serum lactate. Some parameters are categorical as provided, for example, parameters representing if a therapeutic intervention has been applied to the patient or parameters representing a patient's medical history.

At 308, a score, representing a risk that a patient will experience an adverse patient outcome, is generated from at least the set of categorical parameters. In one example, each categorical parameter is assigned a value, and a weighted linear or non-linear combination of the values can be used to calculate the score. In practice, the values assigned to the categorical parameters can serve as the weights for the combination when the combination is linear. It will be appreciated that the biometric parameters can be collected and the score can be calculated on a periodic basis, such that a time series of scores are produced for the patient.

In one example, a parameter representing the risk that the patient will experience the adverse patient outcome can be determined from the time series of scores. In particular, the risk parameter can be calculated from the extremum of the time series of scores over the predefined interval of time. In one implementation, the predefined interval of time can be a period of time immediately preceding the calculation of a last score, or effectively the period of time preceding the calculation of the risk parameter. While this interval can vary with the application, in one example a four hour interval preceding calculation of the risk parameter is used. In one implementation, the risk parameter is a continuous parameter generated as a function of an extremum of the score over the predefined interval. In another implementation, the parameter representing the risk that the patient will experience the adverse patient outcome is a categorical parameter that can assume at least two values. For example, the parameter can represent “normal” and “alarm” states or “normal”, “caution,” and “alarm” states for a given patient. In this instance, generating the parameter representing the risk that the patient will experience the adverse patient outcome from the extremum of the time series of scores over the predefined interval of time can include comparing the extremum of the time series of scores over the predefined interval of time to one or more threshold values and assigning a value for the risk parameter according to this comparison.

FIG. 4 illustrates a method 400 for determining a parameter representing a risk that a patient will experience an adverse patient outcome. At 402, it is determined if defined period of time has passed since a last score for the patient was calculated. If not (N), the method remains at 402. Once the defined period of time has passed (Y), a set of biometric parameters associated with a patient are monitored at 404. At 406, at least one electronic health records (EHR) parameter is retrieved from an EHR database. At 408, a set of categorical parameters is generated from the set of biometric parameters and the at least one EHR parameter according to a predefined rule set. At 410, a score, representing a risk that a patient will experience an adverse patient outcome, is generated for the time period from at least the set of categorical parameters. It will be appreciated that repeated performance of steps 404, 406, 408, and 410 during the method will produce a time series of scores for the patient.

At 412, an extremum, that is, a minimum or a maximum, of the time series of scores over a predefined interval of time is selected. In one example, the predefined interval of time is the preceding four hours. At 414, the parameter representing the risk that the patient will experience the adverse patient outcome is generated from the extremum of the time series of scores over the predefined interval of time. In one implementation, the risk parameter is a categorical parameter generated by comparing the extremum of the time series of scores over the predefined interval of time to at least one threshold value and assigning a value for the parameter to the patient according to the comparison of the extremum to the threshold value or values. The method then returns to 402 to calculate a new score.

FIG. 5 is a schematic block diagram illustrating an exemplary system 500 of hardware components capable of implementing examples of the systems and methods disclosed herein. The system 500 can include various systems and subsystems. The system 500 can be a personal computer, a laptop computer, a workstation, a computer system, an appliance, an application-specific integrated circuit (ASIC), a server, a server BladeCenter, a server farm, etc.

The system 500 can include a system bus 502, a processing unit 504, a system memory 506, memory devices 508 and 510, a communication interface 512 (e.g., a network interface), a communication link 514, a display 516 (e.g., a video screen), and an input device 518 (e.g., a keyboard, touch screen, and/or a mouse). The system bus 502 can be in communication with the processing unit 504 and the system memory 506. The additional memory devices 508 and 510, such as a hard disk drive, server, standalone database, or other non-volatile memory, can also be in communication with the system bus 502. The system bus 502 interconnects the processing unit 504, the memory devices 506-510, the communication interface 512, the display 516, and the input device 518. In some examples, the system bus 502 also interconnects an additional port (not shown), such as a universal serial bus (USB) port.

The processing unit 504 can be a computing device and can include an application-specific integrated circuit (ASIC). The processing unit 504 executes a set of instructions to implement the operations of examples disclosed herein. The processing unit can include a processing core.

The additional memory devices 506, 508, and 510 can store data, programs, instructions, database queries in text or compiled form, and any other information that may be needed to operate a computer. The memories 506, 508 and 510 can be implemented as computer-readable media (integrated or removable), such as a memory card, disk drive, compact disk (CD), or server accessible over a network. In certain examples, the memories 506, 508 and 510 can comprise text, images, video, and/or audio, portions of which can be available in formats comprehensible to human beings.

Additionally or alternatively, the system 500 can access an external data source or query source through the communication interface 512, which can communicate with the system bus 502 and the communication link 514.

In operation, the system 500 can be used to implement one or more parts of a system for assessing the risk of an adverse patient outcome for a patient in accordance with the present invention. Computer executable logic for implementing the diagnostic system resides on one or more of the system memory 506, and the memory devices 508 and 510 in accordance with certain examples. The processing unit 504 executes one or more computer executable instructions originating from the system memory 506 and the memory devices 508 and 510. The term “computer readable medium” as used herein refers to a medium that participates in providing instructions to the processing unit 504 for execution. This medium may be distributed across multiple discrete assemblies all operatively connected to a common processor or set of related processors.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

In the preceding description, specific details have been set forth in order to provide a thorough understanding of example implementations of the invention described in the disclosure. However, it will be apparent that various implementations may be practiced without these specific details. For example, circuits, systems, networks, processes, and other components may be shown as components in block diagram form in order not to obscure the example implementations in unnecessary detail. In other instances, well-known circuits, processes, algorithms, structures, and techniques may be shown without unnecessary detail in order to avoid obscuring the examples. The description of the example implementations will provide those skilled in the art with an enabling description for implementing an example of the invention, but it should be understood that various changes may be made in the function and arrangement of elements without departing from the spirit and scope of the invention. Accordingly, the present invention is intended to embrace all such alterations, modifications, and variations that fall within the scope of the appended claims.

1. A method comprising:
monitoring a set of biometric parameters associated with a patient;
retrieving at least one electronic health records (EHR) parameter from an EHR database;
generating a set of categorical parameters from the set of biometric parameters and the at least one EHR parameter according to a predefined rule set; and
generating a score, representing a risk that a patient will experience an adverse patient outcome, from at least the set of categorical parameters.

2. The method of claim 1, wherein the set of biometric parameters includes at least two of a heart rate, arterial blood pressure, peripheral arterial oxyhemoglobin saturation, and body temperature.

3. The method of claim 1, wherein generating the score comprises generating the score at periodic intervals, such that a time series of scores are produced for the patient.

4. The method of claim 3, further comprising:
selecting an extremum of the time series of scores over a predefined interval of time; and
generating a parameter representing the risk that the patient will experience the adverse patient outcome from the extremum of the time series of scores over the predefined interval of time.

5. The method of claim 4, wherein the parameter representing the risk that the patient will experience the adverse patient outcome is a categorical parameter that can assume at least two values and generating the parameter representing the risk that the patient will experience the adverse patient outcome from the extremum of the time series of scores over the predefined interval of time comprises:
comparing the extremum of the time series of scores over the predefined interval of time to at least one threshold value; and
assigning a value of the at least two values for the parameter representing the risk that the patient will experience the adverse patient outcome to the patient according to the comparison of the extremum of the time series of scores over the predefined interval of time to the at least one threshold value.

6. The method of claim 4, wherein the predetermined interval of time is a period of four hours before the parameter representing the risk that the patient will experience the adverse patient outcome.

7. The method of claim 1, wherein generating the score from at least the set of categorical parameters comprises representing each categorical parameter as an assigned value representing a selected category and calculating the score as a weighted combination of the assigned values representing the set of categorical parameters.

8. The method of claim 7, wherein the weighted combination of the assigned values is a non-linear weighted combination of the assigned values.

9. The method of claim 7, wherein the set of categorical parameters includes a hypothermia parameter, which is assigned a first value of the at least two values when a body temperature of the patient is below a threshold value, and a second value of the at least two values when the body temperature of the patient is above the threshold value.

10. The method of claim 7, wherein the set of categorical parameters includes a tachycardia parameter which is assigned a first value of the at least two values when a heart rate of a patient exceeds a threshold value and a second value of the at least two values when the heart rate of the patient does not exceed the threshold value, wherein the threshold value varies with an age of the patient.

11. The method of claim 1, wherein the set of categorical parameters includes a parameter representing the presence of one of respiratory acidosis and elevated serum lactate.

12. The method of claim 1, wherein the set of categorical parameters includes a parameter representing a therapeutic intervention applied to the patient.

13. A system comprising:
a biometric monitor interface that receives data from at least one monitoring system monitoring a set of biometric parameters associated with a patient;
a network interface that retrieves at least one electronic health records (EHR) parameter from an EHR database;
a feature extractor that generates a set of categorical parameters from the set of biometric parameters and the at least one EHR parameter according to a predefined rule set; and
a predictive model that generates a score, representing a risk that a patient will experience an adverse patient outcome, from at least the set of categorical parameters.

14. The system of claim 13, wherein the predictive model generates the score at periodic intervals, such that a time series of scores are produced for the patient, selects an extremum of the time series of scores over a predefined interval of time, and generates a parameter representing the risk that the patient will experience the adverse patient outcome from the extremum of the time series of scores over the predefined interval of time.

15. The system of claim 14, wherein the predetermined interval of time is a period of four hours before the predictive model generates the parameter representing the risk that the patient will experience the adverse patient outcome.

16. The system of claim 13, wherein the predictive model represents each categorical parameter as an assigned value representing the selected category and calculating the score as a non-linear weighted combination of the assigned values representing the set of categorical parameters.

17. The system of claim 13, wherein a feature extractor generates a first categorical parameter of the set of categorical parameters by comparing a first, continuous biometric parameter of the set of biometric parameters to at least one threshold value and determining a value for the first continuous parameter from the comparison of the first biometric parameter to the at least one threshold value.

18. A method comprising:
monitoring a set of biometric parameters associated with a patient;
retrieving at least one electronic health records (EHR) parameter from an EHR database;
generating, at periodic intervals, a set of categorical parameters from the set of biometric parameters and the at least one EHR parameter according to a predefined rule set;
generating a score for each interval, representing a risk that a patient will experience an adverse patient outcome from at least the set of categorical parameters for the interval to provide a time series of scores;
selecting an extremum of the time series of scores over a predefined interval of time; and
generating a parameter representing the risk that the patient will experience the adverse patient outcome from the extremum of the time series of scores over the predefined interval of time.

19. The method of claim 18, wherein the parameter representing the risk that the patient will experience the adverse patient outcome is a categorical parameter that can assume at least two values and generating the parameter representing the risk that the patient will experience the adverse patient outcome from the extremum of the time series of scores over the predefined interval of time comprises:
comparing the extremum of the time series of scores over the predefined interval of time to at least one threshold value; and
assigning a value of the at least two values for the parameter representing the risk that the patient will experience the adverse patient outcome to the patient according to the comparison of the extremum of the time series of scores over the predefined interval of time to the at least one threshold value.

20. The method of claim 18, wherein the predetermined interval of time is a period of four hours before the parameter representing the risk that the patient will experience the adverse patient outcome.
